(19) United States
(12) Patent Application Publication
(10) Pub. No.: US 2022/0328158 A1
(43) Pub. Date: Oct. 13, 2022

(54) Rehabilitation Support System, Rehabilitation Support Method, and Rehabilitation Support Program

(71) Applicant: Nippon Telegraph and Telephone Corporation, Tokyo (JP)

(72) Inventors: Shin Toyota, Tokyo (JP); Takayuki Ogasawara, Tokyo (JP); Masahiko Mukaino, Toyoake-shi (JP); Eiichi Saitho, Aichi (JP)

(21) Appl. No.: 17/635,649

(22) PCT Filed: Aug. 6, 2020
(86) PCT No.: PCT/JP2020/030223

§ 371 Date: Feb. 15, 2022

(30) Foreign Application Priority Data
Aug. 20, 2019 (JP) 2019-150201

(51) Int. Cl. G16H 20/30; A61B 5/00; A63B 24/00

(52) U.S. Cl. CPC G16H 20/30; A61B 5/7435; A61B 5/7425; A63B 24/0075; A63B 2024/0078

(57) ABSTRACT

In an embodiment, a rehabilitation support system includes: a sensor data acquirer configured to acquire biometric information on a user measured by a sensor; an estimator configured to estimate a state of the user based on the biometric information; a first determiner configured to determine whether the state of the user estimated by the estimator has occurred within a set time, the set time comprising a time slot defined for implementation of a rehabilitation; a first storage device configured to store a mode of a spatiotemporally changing item; a selector configured to select the mode of the spatiotemporally changing item, in accordance with a result of the determination by the first determiner; and a presenter configured to present the mode of the spatiotemporally changing item selected by the selector.

### CROSS-REFERENCE TO RELATED APPLICATIONS

This application is a national phase entry of PCT Application No. PCT/JP2020/030223, filed on Aug. 6, 2020, which application claims priority to Japan Patent Application No. JP2019-150201, filed on Aug. 20, 2019, which applications are hereby incorporated herein by reference.

### TECHNICAL FIELD

The present invention relates to a rehabilitation support system, a rehabilitation support method, and a rehabilitation support program.

### BACKGROUND

With a proper rehabilitation process, patients, elderly people, and the like who need rehabilitation can rehabilitate their physical functions, and achieve their targets in terms of psychological and social aspects regarding their quality of life. Patients requiring rehabilitation may need to aggressively perform rehabilitation over their entire waking hours, to enable them to recover from diseases or the like for example.

Biometric information such as a heart rate and an amount of activity measured by a sensor such as a wearable terminal has been utilized in the fields of sports, and medicine (see, for example, PTL1 and NPL1). For example, PTL1 discloses a technique for analyzing a patient's state of activity more accurately by examining on their lifestyle, based on acceleration measured by a sensor attached to the user.

With the related art, the state of physical activities of the user such as a patient performing rehabilitation (hereinafter, simply referred to as “rehab”) can be recognized and such information can be presented. However, dynamic information has not been presented to the user when he or she works on the rehabilitation.

In view of this, NPL2 proposes an example of a rehabilitation support technique of motivating, based on a record taken when a user implements a certain rehabilitation training, the user toward a rehabilitation.

Unfortunately, the rehabilitation support technique of the related art provides support based on the record taken when a certain rehabilitation training is implemented, and thus with such a technique, it may be difficult to motivate the user who needs rehabilitation toward the rehabilitation over the entirety of his or her waking hours.

### CITATION LIST

### Patent Literature

PTL1: WO 2018/001740

### Non Patent Literature

NPL1: Kasai, Ogasawara, Nakajima, Tsukada, “Development and Application of Functional Material “hitoe” Enabling Measurement of Biometric Information When Worn”, IEICE Communication Society Magazine #41 (June 2017), (Vol. 11, No. 1)

NPL2: Junichi Yamamoto, “Applied Behavior Analysis for Enhancing “Motivation” in Rehabilitation: Use Cases in Physical Therapy”, Japanese Society of Physical Therapy, Physical Therapy Study 41(8), 492-498, 2014

### SUMMARY

### Technical Problem

The embodiments of the present invention have been made to solve the problem described above, and an object of embodiments of the present invention is to provide a rehabilitation support technique with which a user can be more motivated to work on his or her rehabilitation.

### Means for Solving the Problem

In order to solve the problems described above, a rehabilitation support system according to an embodiment of the present invention includes: a sensor data acquisition unit configured to acquire biometric information on a user measured by a sensor; an estimation unit configured to estimate a state of the user based on the biometric information acquired; a first determination unit configured to determine whether the state of the user estimated by the estimation unit has occurred within a set time indicating a time slot defined for implementation of a rehabilitation; a first storage unit configured to store a mode of a spatiotemporally changing item; a selection unit configured to select the mode of the item stored in the first storage unit, in accordance with a result of the determination by the first determination unit; and a presentation unit configured to present the mode of the item selected by the selection unit.

In the rehabilitation support system according to an embodiment of the present invention, the presentation unit may include a display device configured to display an image representing the mode of the item selected by the selection unit.

The rehabilitation support system according to an embodiment of the present invention may further include: a setting unit configured to set the set time for each user, based on statistical data on implementation of the rehabilitation; and a second storage unit configured to store the set time defined for each user, and the first determination unit may determine whether the state of the user estimated by the estimation unit has occurred within the set time defined for the user.

The rehabilitation support system according to an embodiment of the present invention may further include an acceptance unit configured to accept an operation input for setting the mode of the item for each user, and the first storage unit may store the mode of the item set for each user.

The rehabilitation support system according to an embodiment of the present invention may further include a notification unit configured to issue a notification indicating start of the set time.

In the rehabilitation support system according to an embodiment of the present invention, the estimation unit may periodically estimate the state of the user, the rehabilitation support system may further include: a second determination unit configured to determine that the state of the user has transitioned to a state of the user set in advance; and a feedback selection unit configured to select a feedback to the user, in accordance with a result of the determination by the second determination unit, the state of the user may include a plurality of different states corresponding to different exercise loads, and the feedback selection unit may select as the feedback, the mode of the spatiotemporally changing item.

In order to solve the problems described above, a rehabilitation support method according to an embodiment of the present invention includes: a first step of acquiring biometric information on a user measured by a sensor; a second step of estimating a state of the user based on the biometric information acquired in the first step; a third step of determining whether the state of the user estimated in the second step has occurred within a set time indicating a time slot defined for implementation of a rehabilitation; a fourth step of selecting a mode of a spatiotemporally changing item stored in a first storage unit, in accordance with a result of the determination in the third step; and a fifth step of presenting the mode of the item selected in the fourth step.

In order to solve the problems described above, a rehabilitation support program according to an embodiment of the present invention causes a computer to perform: a first step of acquiring biometric information on a user measured by a sensor; a second step of estimating a state of the user based on the biometric information acquired in the first step; a third step of determining whether the state of the user estimated in the second step has occurred within a set time indicating a time slot defined for implementation of a rehabilitation; a fourth step of selecting a mode of a spatiotemporally changing item stored in a first storage unit, in accordance with a result of the determination in the third step; and a fifth step of presenting the mode of the item selected in the fourth step.

### Effects of Embodiments of the Invention

According to embodiments of the present invention, whether the estimated state of the user has occurred within a time slot defined for implementation of a rehabilitation is determined and the mode of the spatiotemporally changing item is selected in accordance with a result of the determination. Therefore, a user can be more motivated to work on his or her rehabilitation.

### BRIEF DESCRIPTION OF THE DRAWINGS

FIG. 1 is a block diagram illustrating a functional configuration of a rehabilitation support system according to a first embodiment of the present invention.

FIG. 2 is a block diagram illustrating a configuration of a data analysis unit according to the first embodiment.

FIG. 3 is a block diagram illustrating an example of a computer configuration that implements the rehabilitation support system according to the first embodiment.

FIG. 4 is a flowchart illustrating an operation of the rehabilitation support system according to the first embodiment.

FIG. 5 is a diagram illustrating an outline of an example of a configuration of the rehabilitation support system according to the first embodiment.

FIG. 6 is diagram illustrating a display example of rehabilitation support information according to the first embodiment.

FIG. 7 is a diagram illustrating an example of a mode of an image according to the first embodiment.

FIG. 8 is a block diagram illustrating an example of a configuration of the rehabilitation support system according to the first embodiment.

FIG. 9 is a sequence diagram illustrating an operation of the rehabilitation support system according to the first embodiment.

FIG. 10 is a block diagram illustrating a configuration of a selection unit according to a second embodiment.

FIG. 11 is a diagram illustrating a set time according to the second embodiment.

FIG. 12 is a flowchart illustrating an operation of the rehabilitation support system according to the second embodiment.

FIG. 13 is a block diagram illustrating a configuration of the rehabilitation support system according to a third embodiment.

FIG. 14 is a diagram illustrating an operation of the rehabilitation support system according to the third embodiment.

FIG. 15 is a diagram illustrating a set time according to the third embodiment.

FIG. 16 is a block diagram illustrating a configuration of the rehabilitation support system according to a fourth embodiment.

FIG. 17 is a block diagram illustrating a configuration of a selection unit according to a fifth embodiment.

FIG. 18 is a flowchart illustrating an operation of the rehabilitation support system according to the fifth embodiment.

### DETAILED DESCRIPTION OF ILLUSTRATIVE EMBODIMENTS

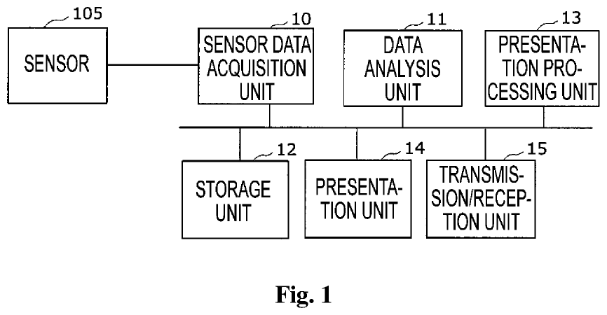

Preferred embodiments of the present invention will be described below in detail with reference to FIGS. 1 to 18.

### First Embodiment

First, an outline of a configuration of a rehabilitation support system according to a first embodiment of the present invention will be described. FIG. 1 is a block diagram illustrating a functional configuration of a rehabilitation support system. The rehabilitation support system acquires biometric information on a user measured by a sensor 105, estimates a state of the user involved in the rehabilitation performed by the user, and selects and presents a mode of a spatiotemporally changing item in accordance with the progress of the rehabilitation based on a result the estimation.

### Functional Block of Rehabilitation Support System

The rehabilitation support system includes a sensor data acquisition unit 10 that acquires data from the sensor 105, a data analysis unit 11, a storage unit 12, a presentation processing unit 13, a presentation unit 14, and a transmission/reception unit 15.

The sensor data acquisition unit 10 acquires the biometric information on the user measured by the sensor 105. More specifically, when an acceleration sensor, as the sensor 105, is attached to the user, the sensor data acquisition unit 10 converts an analog acceleration signal measured by the acceleration sensor into a digital signal at a predetermined sampling rate. The biometric information measured by the sensor data acquisition unit 10 is stored in the storage unit 12, which is described later, in association with a measurement time point.

The sensor data acquisition unit 10 may acquire, as the biometric information on the user, angular velocity, light, electromagnetic waves, temperature and humidity, pressure, position information, sound, concentration, voltage, resistance, and the like, in addition to acceleration. Furthermore, the sensor data acquisition unit 10 can acquire, as the biometric information on the user, cardiac electrical activity, myoelectric activity, blood pressure, intrabody gas exchanged by respiration, body temperature, pulse, and brainwave obtained from these physical quantities.

Furthermore, in addition to the biometric information on the user, the sensor data acquisition unit 10 may acquire external environment data on the location of the user. The external environment data includes, for example, room temperature, ambient temperature, humidity, and the like at the location where the user is located. Note that the sensor data acquisition unit 10 may acquire the biometric information on the user from each of a plurality of the sensors 105 measuring the biometric information on the user.

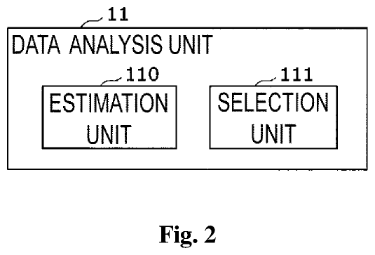

The data analysis unit 11 analyzes the biometric information on the user acquired by the sensor data acquisition unit 10 to estimate the state of the user, and selects the mode of the spatiotemporally changing item in accordance with the estimated state of the user. As illustrated in FIG. 2, the data analysis unit 11 includes an estimation unit 110 and a selection unit 111.

The estimation unit 110 calculates the state of the user from the biometric information on the user acquired by the sensor data acquisition unit 10. The state of the user refers to the posture, coordinates, speed, speech, breathing, walking, seating, driving, sleeping, body movement, stress, and the like involved in the rehabilitation performed by the user. Furthermore, the calculation may be performed to obtain other results which are information indicating quantity such as the magnitude, frequency, increase/decrease, duration, and accumulation of such states.

Specifically, the estimation unit 110 may estimate the state of the user using, for example, an out-of-bed state and a lying state estimated using the acceleration of the user described in PTL1. With the state of the user estimated by the estimation unit 110, the progress of the user's rehabilitation can be recognized.

The estimation unit 110 estimates the state of the user based on the biometric information on the user acquired over a period of time from the start of the measurement with the sensor 105 attached to the user to the current measurement time point. The result of estimating the state of the user by the estimation unit 110 is stored in the storage unit 12 together with time information.

The selection unit 111 selects the spatiotemporally changing mode of the item stored in the storage unit 12, in accordance with the state of the user estimated by the estimation unit 110. More specifically, the selection unit 111 can select a presented image representing the mode of the spatiotemporally changing item, by using a history of the state within any period from the start of the measurement of the biometric information on the user performing the rehabilitation. For example, a scene of a movie is selected using a state history, estimated by the estimation unit 110, indicating a period during which the user is in the out-of-bed state.

Alternatively, the selection unit 111 can select the mode of the spatiotemporally changing image, in accordance with the current state of the user which is, for example the state of the user estimated at the current time point, without using a history of the user's state.

The spatiotemporally changing item is information presented to the user as rehabilitation support information. Hereinafter, the spatiotemporally changing item and information including the same may be referred to simply as the rehabilitation support information.

As the spatiotemporally changing item, for example, movie, sound, text, and combinations thereof can be used that represent the progress of the rehabilitation relative to a target value set in accordance with a frequency of awakening performed by the user as rehabilitation or the out-of-bed time. Further, information presented in a form perceptible by the user such as vibration, heat, light, wind, or the like may be added to the movie or the like. Furthermore, a stereoscopic image such as a hologram may be used as the image.

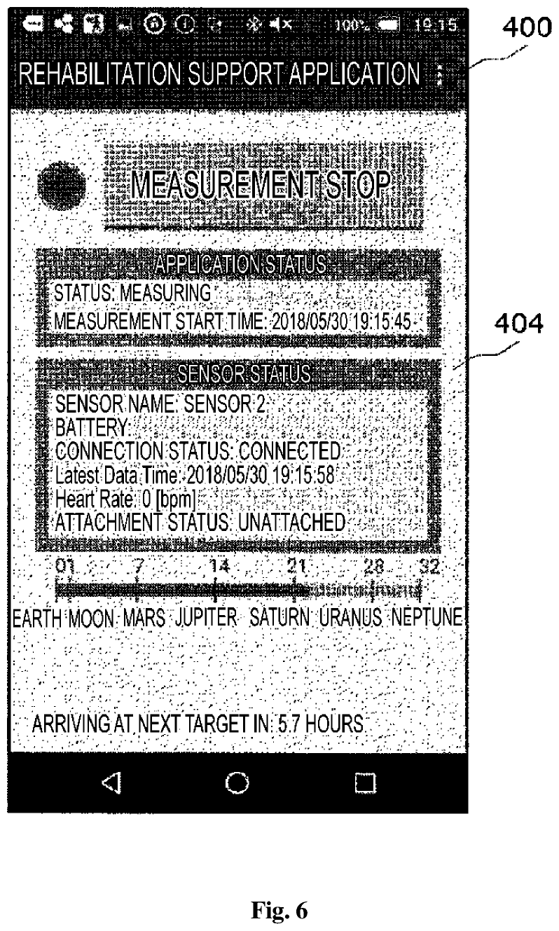

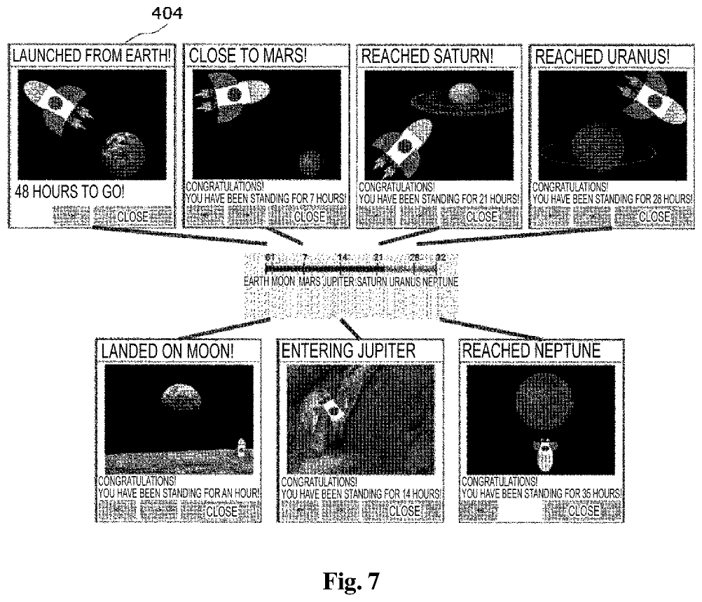

Specifically, as illustrated in FIGS. 6 and 7, an image of a space craft traveling in space can be used as the spatiotemporally changing item presented as the rehabilitation support information. For example, when a user who is often in the lying state performs rehabilitation such as getting out of bed, the selection unit 111 selects image and text information indicating that the space craft has been launched from the earth, at the point when the measurement by the sensor 105 starts. Thereafter, the mode of the presented image is selected with the arrival point of the space craft switched, each time the out-of-bed time reaches a certain period of time, to Mars, Jupiter, Saturn, and Neptune which is the final destination farthest from the earth, in this order.

The rehabilitation support information illustrated in FIGS. 6 and 7 is an example, and the mode of the rehabilitation support information selected by the selection unit 111 is not limited to scenes of an animation of the space craft traveling. For example, the progress of the rehabilitation started by the user reaching a target set in advance may be represented by a movie or a still image such as an image indicating a process of building a building or a monument, an image indicating the growth of a plant or an animal, an image indicating actions of a character such as a person, and an animation indicating a musical performance, a competition held, solution of a puzzle, acquisition of honors and reward, as well as by text information, sound, and the like added to the image.

Information indicating the mode of the spatiotemporally changing item selected by the selection unit 111 is input to the presentation processing unit 13.

The storage unit (first storage unit) 12 stores a mode of the spatiotemporally changing item. More specifically, the storage unit 12 can store in advance an image that changes in accordance with the progress of the rehabilitation described above. Information indicating the progress of the rehabilitation is associated with each image. For example, information indicating that the out-of-bed state has continued for a total time of an hour is stored in association with a mode of an image of the space craft reaching the moon.

The storage unit 12 stores time series data on biometric information on the user acquired by the sensor data acquisition unit 10. The storage unit 12 stores a history of the state of the user estimated by the estimation unit 110. The state of the user thus estimated is stored in the storage unit 12 together with the measurement time of the biometric information on which the state is based.

The presentation processing unit 13 generates the image presented by the presentation unit 14, based on the information indicating the mode of the spatiotemporally changing item selected by the selection unit 111. More specifically, the presentation processing unit 13 generates a movie presented as the rehabilitation support information, by using a still image of a format such as jpg, png, or bpm or a movie of a format such as gif, flash, or mpg as an image of a format set in advance. The presentation processing unit 13 can also generate rehabilitation support information such as sound or text presented in combination with the movie, as described above.

The presentation unit 14 causes a display device 109 described later to present the rehabilitation support information generated by the presentation processing unit 13. The presentation unit 14 switches the image presented by the display device 109 based on a signal from the presentation processing unit 13. The presentation unit 14 may present external environment data, acquired along with biometric information by the sensor data acquisition unit 10, together with the rehabilitation support information.

The transmission/reception unit 15 receives sensor data indicating the biometric information on the user measured by the sensor 105. The transmission/reception unit 15 may convert information indicating the rehabilitation support information determined by the data analysis unit 11 according to a predetermined communication standard, and transmit the information to the presentation unit 14 connected to the communication network.

### Configuration of Computer for Rehabilitation Support System

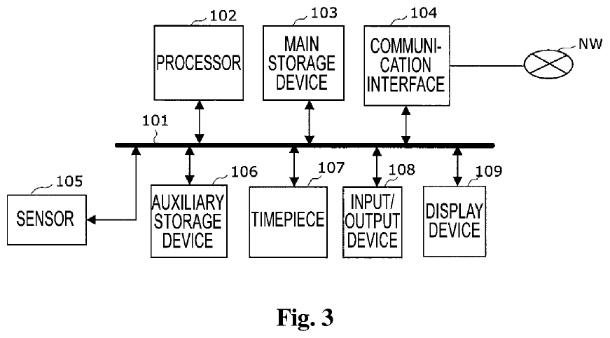

Next, an example of a computer configuration for achieving the rehabilitation support system having the functions described above will be described with reference to FIG. 3.

As illustrated in FIG. 3, the rehabilitation support system may be achieved by, for example, a computer including a processor 102, a main storage device 103, a communication interface 104, an auxiliary storage device 106, a timepiece 107, and an input/output device 108 connected to each other through a bus 101, and a program for controlling these hardware resources. The rehabilitation support system has, for example, the display device 109 provided therein and the sensor 105 provided outside the rehabilitation support system connected to each other via the bus 101.

The main storage device 103 stores in advance programs for the processor 102 to perform various controls and calculations. The processor 102 and the main storage device 103 achieve the functions of the rehabilitation support system including the data analysis unit 11 as illustrated in FIG. 1 and FIG. 2.

The communication interface 104 is an interface circuit for communicating with various external electronic devices via a communication network NW.

Examples of the communication interface 104 include an arithmetic interface and an antenna that comply with wireless data communication standards such as LTE, 3G, a wireless LAN, and Bluetooth (trade name). The transmission/reception unit 15 illustrated in FIG. 1 is achieved by the communication interface 104.

The sensor 105 includes, for example, a heart rate meter, an electrocardiograph, a blood pressure meter, a pulse rate meter, a respiration sensor, a thermometer, a brainwave sensor, and the like. More specifically, the sensor 105 is achieved by a three-axis acceleration sensor, a microwave sensor, a pressure sensor, a current meter, a voltmeter, a thermo-hygrometer, a concentration sensor, a photosensor, or a combination thereof.

The auxiliary storage device 106 is configured of a readable and writable storage medium, and a drive device for reading or writing various types of information such as programs or data from or to the storage medium. A hard disk or a semiconductor memory such as a flash memory can be used as a storage medium in the auxiliary storage device 106.

The auxiliary storage device 106 includes a storage region for storing the biometric information measured by the sensor 105 and a program storage region for storing a program for the rehabilitation support system to implement analysis processing on the biometric information. The storage unit 12 illustrated in FIG. 1 is achieved by the auxiliary storage device 106. Further, for example, a backup area for backing up the data, programs, and the like described above may be provided.

The timepiece 107 includes a built-in timepiece or the like of the computer and clocks a time. Alternatively, the timepiece 107 may acquire time information from a time server not illustrated in the drawing. The time information obtained by the timepiece 107 is recorded in association with the state of the user estimated. The time information obtained by the timepiece 107 is used for sampling of the biometric information or the like.

The input/output device 108 includes an I/O terminal that receives a signal from an external device such as the sensor 105 and the display device 109 and outputs a signal to an external device.

The display device 109 is implemented by a liquid crystal display or the like. The display device 109 achieves the presentation unit 14 illustrated in FIG. 1.

### Rehabilitation Support Method

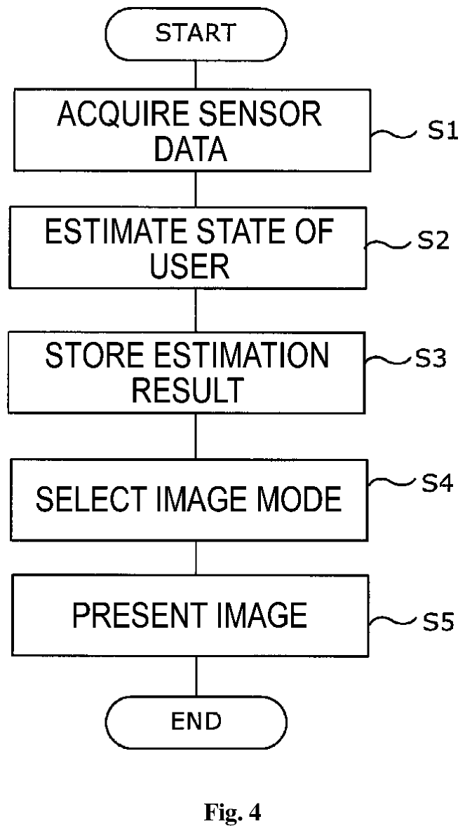

Next, the operation of the rehabilitation support system configured as described above will be described with reference to a flowchart of FIG. 4. First, the following processing is executed in a state where the sensor 105 is attached to the user for example.

The sensor data acquisition unit 10 acquires the biometric information on the user measured by the sensor 105 via the transmission/reception unit 15 (step S1). The acquired biometric information is accumulated in the storage unit 12. Note that the sensor data acquisition unit 10 can execute processing of remove the noise in the biometric information acquired, and converting the analog signal of the biometric information into a digital signal.

Next, the estimation unit 110 estimates the state of the user based on the biometric information on the user acquired by the sensor data acquisition unit 10 (step S2). For example, the estimation unit 110 estimates that the user is in the out-of-bed state from the data indicating the acceleration of the user acquired by the sensor data acquisition unit 10. The result of the estimation by the estimation unit 110 is stored in the storage unit 12 along with the time information (step S3).

Thereafter, the selection unit 111 selects a mode of the spatiotemporally changing item to be presented as the rehabilitation support information, by using a history within any period from the start of measurement of sensor data based on which the state of the user that performs the rehabilitation is estimated by the estimation unit 110 (step S4). For example, as illustrated in FIG. 6 and FIG. 7, a case is considered in which a user who is often in the lying state gets out of bed as rehabilitation. In this case, a movie of the space craft that has departed from the earth traveling in space to reach Neptune is used as the rehabilitation support information indicating the length of time during which the user is in the out-of-bed state. The selection unit 111 selects, using a history of the state of the user stored in the storage unit 12, an image of a planet, which is a passing point of the space craft, corresponding to the time during which the user is in the out-of-bed state.

Next, the presentation unit 14 causes the display device 109 to display the mode of the spatiotemporally changing item selected by the selection unit 111, as the rehabilitation support information (step S5). More specifically, the presentation processing unit 13 generates an image, sound, and text corresponding to the mode of the spatiotemporally changing image selected by the selection unit 111. The presentation unit 14 outputs the image of the mode generated by the presentation processing unit 13 as the rehabilitation support information.

### Specific Configuration of Rehabilitation Support System

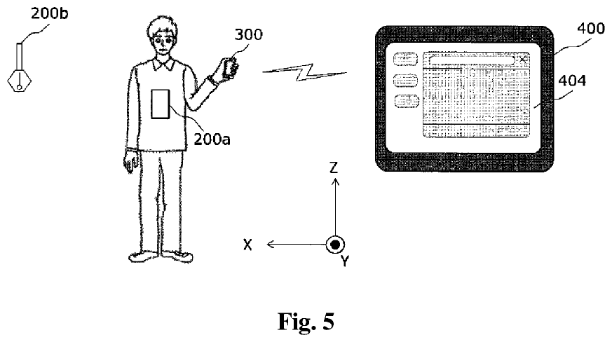

Next, an example of a specific configuration of the rehabilitation support system according to an embodiment of the present invention will be described with reference to FIG. 5 to FIG. 8.

As illustrated in FIG. 5, for example, the rehabilitation support system includes a sensor terminal 200a attached to the user performing rehabilitation, a sensor terminal 200b that measures external environment data on the location of the user, a relay terminal 300, and an external terminal 400. All or any of the sensor terminals 200a and 200b, the relay terminal 300, and the external terminal 400 includes the functions included in the rehabilitation support system such as the data analysis unit 11 illustrated in FIG. 1 and FIG. 2. The following description is given under an assumption that the relay terminal 300 includes the data analysis unit 11 illustrated in FIG. 1, and the rehabilitation support information is presented on the external terminal 400.

### Functional Block of Sensor Terminal

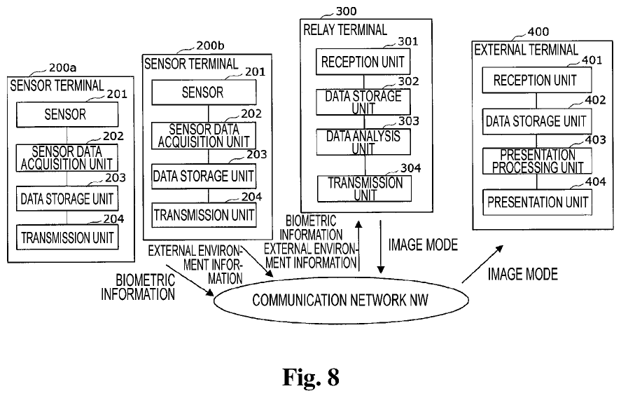

The sensor terminals 200a and 200b each include a sensor 201, a sensor data acquisition unit 202, a data storage unit 203, and a transmission unit 204 as illustrated in FIG. 8. For example, the sensor terminal 200a is placed on the trunk of the body of the user, and measures biometric information such as acceleration and the body temperature. The sensor terminal 200b measures external environment data such as humidity and temperature at a location of the user. The sensor terminals 200a and 200b transmit the biometric information on the user and the external environment data to the relay terminal 300 through the communication network NW.

The sensor 201 is achieved by the three-axis acceleration sensor and the like for example. Regarding the three axes of the acceleration sensor included in the sensor 201, the X axis is provided in parallel with the right-left direction of the body, the Y axis is provided in parallel with the front-back direction of the body, and the Z axis is provided in parallel with the up-down direction of the body, for example, as illustrated in FIG. 5. The sensor 201 corresponds to the sensor 105 described in FIG. 1.

The sensor data acquisition unit 202 acquires the biometric information and external environment data measured by the sensor 201. The sensor data acquisition unit 202 performs noise removal and sampling processing on the biometric information acquired, and obtains time series data on the biometric information and the like of the digital signal. The sensor data acquisition unit 202 corresponds to the sensor data acquisition unit 10 described in FIG. 1.

The data storage unit 203 stores the biometric information and the external environment data measured by the sensor 201 and the time series data on the biometric information indicated by the digital signal obtained by the processing by the sensor data acquisition unit 202 and the like. The data storage unit 203 corresponds to the storage unit 12 (FIG. 1).

The transmission unit 204 transmits the biometric information and the external environment data stored in the data storage unit 203 to the relay terminal 300 through the communication network NW. The transmission unit 204 includes a communication circuit for performing wireless communication in compliance with wireless data communication standards such as LTE, 3G, a wireless local area network (LAN), or Bluetooth (trade name) for example. The transmission unit 204 corresponds to the transmission/reception unit 15 (FIG. 1).

### Functional Block of Relay Terminal

The relay terminal 300 includes a reception unit 301, a data storage unit 302, a data analysis unit 303, and a transmission unit 304. The relay terminal 300 analyzes the biometric information on the user received from the sensor terminal 200a. Furthermore, the relay terminal 300 estimates the state of the user who performs the rehabilitation based on the biometric information on the user. Furthermore, the relay terminal 300 selects and determines the corresponding rehabilitation support information based on the state of the user estimated. Information indicating the determined rehabilitation support information is transmitted to the external terminal 400.

The relay terminal 300 is implemented by a smart phone, a tablet, a laptop computer, a gateway, or the like.

The reception unit 301 receives the biometric information and the external environment data from the sensor terminals 200a and 200b through the communication network NW. The reception unit 301 corresponds to the transmission/reception unit 15 (FIG. 1).

The data storage unit 302 stores the biometric information on the user received by the reception unit 301, the external environment data, and history of the state of the user within the measurement period estimated by the data analysis unit 303. The data storage unit 302 corresponds to the storage unit 12 (FIG. 1).

The data analysis unit 303 analyzes the biometric information on the user received by the reception unit 301, estimates a state of the user involved in the rehabilitation performed by the user, and selects the mode of the item such as a scene of the spatiotemporally changing movie in accordance with the progress of the rehabilitation based on the estimation result. The data analysis unit 303 corresponds to the data analysis unit 11 including the estimation unit 110 and the selection unit 111 described in FIGS. 1 and 2.

The transmission unit 304 transmits information indicating the mode of the spatiotemporally changing item selected by the data analysis unit 303 to the external terminal 400 through the communication network NW. The transmission unit 304 corresponds to the transmission/reception unit 15 (FIG. 1).

### Functional Block of External Terminal

The external terminal 400 includes a reception unit 401, a data storage unit 402, a presentation processing unit 403, and a presentation unit 404. The external terminal 400 generates and presents the rehabilitation support information based on the information received from the relay terminal 300 through the communication network NW.

Similar to the relay terminal 300, the external terminal 400 is implemented by a smart phone, a tablet, a laptop computer, a gateway, or the like. The external terminal 400 includes the display device 109 that generates and displays an image corresponding to the mode of the image of the received rehabilitation support information. Note that, in addition to the display device 109, the rehabilitation support information may be presented using a sound output device, a light source, or the like not illustrated in the drawings.

The reception unit 401 receives information indicating the mode of the spatiotemporally changing image, presented as the rehabilitation support information, from the relay terminal 300 through the communication network NW. The reception unit 401 corresponds to the transmission/reception unit 15 (FIG. 1).

The data storage unit 402 stores the mode of the spatiotemporally changing item. The data storage unit 402 corresponds to the storage unit 12 (FIG. 1).

The presentation processing unit 403 reads an image to be presented as the rehabilitation support information from the data storage unit 402, and outputs the image. The presentation processing unit 403 can generate an image of the mode corresponding to the state of the user, such as the progress of the rehabilitation performed by the user, and control the display format of the rehabilitation support information. The presentation processing unit 403 may read a material such as an image, movie, sound, or the like set in advance, and may encode a result of editing including: combining the moving to be presented with sound or the like; setting playback speed; and processing using an effect filter. The presentation processing unit 403 corresponds to the presentation processing unit 13 illustrated in FIG. 1.

The presentation unit 404 outputs, as the rehabilitation support information, a spatiotemporally changing image of the selected mode, based on an instruction from the presentation processing unit 403. The presentation unit 404 may display the scene of the movie and the text information corresponding to the progress of the rehabilitation performed by the user on the display device 109, or output sound from a speaker (not illustrated) included in the external terminal 400. In addition, the presentation unit 404 can present the rehabilitation support information by a method perceptible by the user such as vibration, light, and stimulation. The presentation unit 404 may present information about an external environment, such as the temperature measured by the sensor terminal 200b, together with the image representing the selected scene of the movie. The presentation unit 404 corresponds to the presentation unit 14 described in FIG. 1.

As described above, the rehabilitation support system according to an embodiment of the present invention has a configuration in which the functions illustrated in FIGS. 1 and 2 are distributed among the sensor terminals 200a and 200b, the relay terminal 300, and the external terminal 400. The rehabilitation support system according to an embodiment of the present invention is configured to execute, in a distributed manner, processing of: estimating the state of the user from the acquired biometric information on the user; selecting the mode of the item such as the spatiotemporally changing image in accordance with the state of the user; and generating and presenting the image of the selected mode.

### Operating Sequence of Rehabilitation Support System

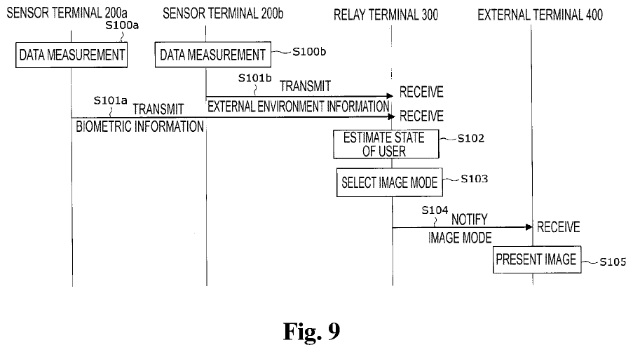

Next, operations of the rehabilitation support system having the above-described configuration will be described using the sequence diagram of FIG. 9.

As illustrated in FIG. 9, first, the sensor terminal 200a is attached to the user and measures biometric information such as three-axis acceleration for example (step S100a). The sensor terminal 200a acquires a digital signal of the biometric information measured, and removes noise as necessary. Next, the sensor terminal 200a transmits the biometric information to the relay terminal 300 through the communication network NW (step S101a).

On the other hand, the sensor terminal 200b is installed at a location where the user is located, and measures data indicating an external environment such as the temperature (step S100b). The information indicating the measured external environment is transmitted to the relay terminal 300 through the communication network NW (step S101b).

Then, upon receiving the biometric information from the sensor terminal 200a, the relay terminal 300 estimates the state of the user based on the biometric information (step S102). More specifically, the data analysis unit 303 of the relay terminal 300 calculates the state of the user involved in the rehabilitation from the biometric information, and records the biometric information together with the time information indicating the time when the biometric information, on which the state of the user is based, is measured.

Next, the data analysis unit 303 selects the mode of the spatiotemporally changing item in accordance with the state of the user estimated in step S102 (step S103). Then, the relay terminal 300 transmits information indicating the selected mode of the item to the external terminal 400 through the communication network NW (step S104). In this process, the information indicating the external environment measured by the sensor terminal 200b is also transmitted to the external terminal 400. Upon receiving the information indicating the mode of the item, the external terminal 400 performs the presentation processing for the item to be presented as the rehabilitation support information (step S105).

Now, an example of how the rehabilitation support information is presented on the external terminal 400 will be described with reference to FIG. 6 and FIG. 7. As illustrated in FIG. 6, the biometric information measured by the sensor terminal 200a, for example, the measurement start time indicating when the acceleration of the user is measured, and the measurement time indicating when the latest data is measured are displayed on the external terminal 400. FIG. 6 and FIG. 7 illustrate an example in which a user is engaged in the rehabilitation of getting out of bed. The movie displayed with the scenes switching to show a story of a space craft launched from the earth to reach Neptune as the target destination, which represents the progress from the start of the rehabilitation performed by the user to the achievement the target level.

As illustrated in FIG. 6 and FIG. 7, on the display screen of the external terminal 400, an image of first arriving at the moon is displayed, when the out-of-bed time according to the history recorded in the out-of-bed state of the user is an hour. Then, when the out-of-bed state of the user falls within a range of 7 hours or longer and shorter than 14 hours, the displayed image is switched to an image of arrival at Mars. When the out-of-bed state of the user falls within a range of 14 hours or longer and shorter than 21 hours, the displayed image is switched to an image of arrival at Jupiter. When the out-of-bed state of the user reaches 21 hours, the displayed image is switched to an image of arrival at Saturn. When the out-of-bed state of the user reaches 28 hours, the displayed image is switched to an image of arrival at Uranus. When the out-of-bed state of the user reaches 35 hours, the displayed image is switched to an image of arrival at Neptune. In addition to the images indicating these points of arrival, the text information indicating the progress of the rehabilitation is presented.

In this manner, the scene of the movie presented switches to the next one in accordance with the progress of the rehabilitation, that is, each time the duration of the out-of-bed state of the user exceeds a duration set in advance. The information indicating the external environment such as the temperature is presented together with such a movie.

As described above, the rehabilitation support system according to the first embodiment estimates the state of the user involved in the rehabilitation based on the biometric information on the user measured by the sensor 105, and selects and presents the mode of the spatiotemporally changing image in accordance with the state of the user thus estimated. Thus, the user can easily recognize the progress of the rehabilitation, to be more motivated toward the rehabilitation.

### Second Embodiment

Next, a second embodiment of the present invention will be described. In the following description, the same components as those in the first embodiment described above will be denoted by the same reference signs and description thereof will be omitted.

In the case described in the first embodiment, the data analysis unit 11 estimates, from the biometric information acquired by the sensor data acquisition unit 10, a state of a user involved in the rehabilitation, such as the out-of-bed state, for example, and selects and presents the mode of the spatiotemporally changing image, based on the result of the estimation. On the other hand, in the second embodiment, a selection unit 111A selects the mode of the spatiotemporally changing image, by using information on the occurrence time of the state of the user estimated.

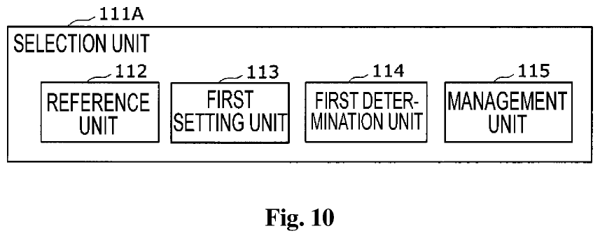

As illustrated in FIG. 10, the selection unit 111A included in the rehabilitation support system according to the second embodiment is different from that in the first embodiment in that the selection unit 111A includes a reference unit 112, a first setting unit 113, a first determination unit 114, and a management unit (selection unit) 115. Hereinafter, components different from those of the first embodiment will be mainly described.

The reference unit 112, with reference to the timepiece 107 that measures the current time, acquires the time when the state of the user occurs.

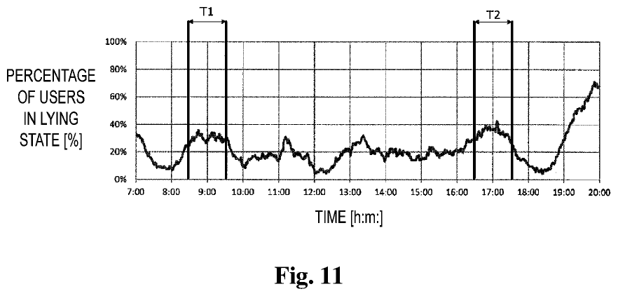

The first setting unit 113 sets a time point or a time slot defined for the implementation of the rehabilitation. As the time or time slot defined for the implementation of the rehabilitation, a statistically determined time, a time slot, and the like can be used. Specifically, FIG. 11 illustrates percentages of those who are in the lying state among 106 hospitalized patients requiring the rehabilitation of getting out of bed, within 24 hours (a day) of their lives. In the example illustrated in FIG. 11, a time slot T1 that is from 8:30 to 9:30 and a time slot T2 that is from 16:30 to 17:30 are set as time slots defined for the implementation of the rehabilitation in a day. In this specific example, the time slots T1 and T2 are set as time slots during which the users who are often in the lying state are particularly desired to be prompted to perform the rehabilitation of getting out of bed.

The first setting unit 113 may use, as set time, a time slot during which a state where the number of users who are in the lying state is particularly large (the time during which 30% of all the users are in the lying state for example) continues for a certain amount of time during the waking hours (7:00 to 20:00) within 24 hours may be used as illustrated in FIG. 11, to prompt the users who are often in the lying state to reduce their time in the lying state and actively perform the rehabilitation. In this case, for example, a time slot following during which a state where many users are in the lying state continues for an hour or more may be used as the set time.

The time or time slot set by the first setting unit 113 may be a day, a day of the week, a week, a month, a year, or the like, and the number of the times or time slots can be set as desired. The first setting unit 113 can set the set time by receiving an external operation input. Alternatively, the first setting unit 113 may estimate the set time using statistical data, related to the rehabilitation, stored in the storage unit 12 in advance.

The first determination unit 114 determines whether the time acquired by the reference unit 112 by referring to the timepiece 107 is included in the time or the time slot set by the first setting unit 113. For example, in the example of FIG. 11, when the time when the state of the user, for example, the out-of-bed state has occurred, acquired by the reference unit 112, is 9:00, the time is determined to be included in the set time slot T1.

When the time when the user enters the out-of-bed state acquired by the reference unit 112 is 10:00, the time is determined not to be in any of the time slots T1 and T2. The result of the determination by the first determination unit 114 is transmitted to the management unit 115.

Based on the result of the determination by the first determination unit 114, the management unit 115 selects the mode of the spatiotemporally changing item. More specifically, when the first determination unit 114 determines that the time when the state of the user has occurred is included in the set time slot, the management unit 115 selects the mode of the spatiotemporally changing item that can more motivate the user to perform the rehabilitation he or she is currently engaged in.

Specifically, a case is considered in which a movie of a space craft traveling in space as described above is presented as the rehabilitation support information. The management unit 115 can select an image in which, for example, the space craft with a booster is traveling when the time when the user has performed the rehabilitation of getting out of bed is included in the set time slot T1. On the other hand, when the time when the user has performed the rehabilitation is not included in any of the set time slots T1 and T2, an image of a normal space craft may be selected. For example, in a case where the user has performed a rehabilitation of getting out of bed or of walking involving a larger exercise load in the time slots T1 and T2 in which the user is desired to be prompted to get out of bed, a mode of an image of reward information that further motivates and prompts the user to perform the rehabilitation is selected and presented as the rehabilitation support information.

When the user is not performing the rehabilitation and is in the lying state in the time slots T1 and T2 in which the user is desired to be prompted to perform the rehabilitation of getting out of bed, a mode of an image prompting the user to start the rehabilitation may be selected. In this case, an image of a mode is selected that can give the user a reason to start the rehabilitation. For example, a configuration can be adopted in which a special image is displayed when the user performs the rehabilitation of getting out of bed.

The storage unit 12 stores, in association with a mode of a reward image, a condition that has to be satisfied for the mode of the reward image to be selected.

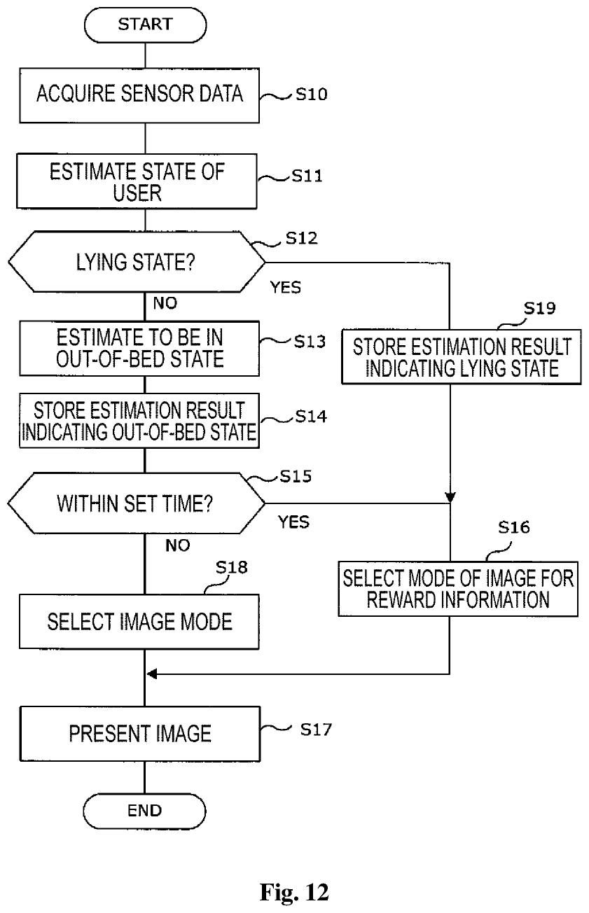

Next, the operation of the rehabilitation support system of the present embodiment configured as described above will be described with reference to a flowchart of FIG. 12. First, the following processing is executed in a state where the sensor 105 is attached to the user. Furthermore, a specific example where the user who is often in the lying state is prompted to perform the rehabilitation of getting out of bed will be described.

The sensor data acquisition unit 10 acquires the biometric information on the user measured by the sensor 105 via the transmission/reception unit 15 (step S10). The acquired biometric information is accumulated in the storage unit 12. Note that the sensor data acquisition unit 10 can execute processing of remove the noise in the biometric information acquired, and converting the analog signal of the biometric information into a digital signal.

Next, the estimation unit 110 estimates the state of the user based on the biometric information on the user acquired by the sensor data acquisition unit 10 (step S11). For example, the estimation unit 110 estimates that the user is in the lying state or the out-of-bed state from the data indicating the acceleration of the user acquired by the sensor data acquisition unit 10.

When the estimation state of the user is the lying state (step S12: YES), the state of the user is stored in the storage unit 12 along with the time information (step S19). Then, the management unit 115 selects the mode of the spatiotemporally changing image, corresponding to reward information stored in the storage unit 12 in advance (step S16). More specifically, the management unit 115 selects reward information prompting the user in the lying state to start the rehabilitation of getting out of bed or the like, which is, for example, an image of a mode indicating that the user will receive a point if he or she performs the rehabilitation.

On the other hand, when the user is determined to be in the out-of-bed state (step S13), the state of the user is stored in the storage unit 12 along with the time information (step S14). Then, when time when the out-of-bed state of the user occurs is determined to be included in the set time slot by the first determination unit 114 (step S15: YES), the management unit 115 selects the spatiotemporally changing image that represents the reward information stored in the storage unit 12 (step S16). For example, reward information of doubling the traveling speed of the space craft or of giving a special point is selected.

As described above, as the reward information, a mode of an image is selected to be different between a case where the user is not performing the rehabilitation and a case where the user is performing the rehabilitation in the time slot in which the rehabilitation is particularly recommended. Furthermore, depending on the action of the user in the set time slot, an image of a special mode is selected, and the progress of the game or story represented by the image changes. Depending on the state of the user estimated in the set time slot, progression speed of a game, a story, or the like represented by a movie or the like increases from that in the normal state. For example, a configuration may be adopted where the time it takes for the image to be switched is halved. Note that the normal state corresponds to a case in which the mode of the image is selected in accordance with the state of the user when the user is in the same state in a time slot other than the set time slot.

Then, the presentation unit 14 causes the display device 109 to display an image of the selected mode representing the reward information as the rehabilitation support information (step S17). For example, in a case where an image displayed is supposed to be switched to an image of the space craft reaching Mars when the time is within the set time in step S15, the presentation unit 14 may display an image of the space craft with a booster passing through Mars and is close to Jupiter.

When the user is estimated to be in the lying state without performing the rehabilitation, the presentation unit 14 may display an image of a mode indicating that the user is given a point when he or she starts the rehabilitation or the like.

On the other hand, when the time when the out-of-bed state occurs by the user is not within a range of the set time in step S15 (step S15: NO), the selection unit 111A selects the mode of the spatiotemporally changing image, based on the state of the user as in the normal case (step S18). Then, the presentation unit 14 similarly switches the image and causes the display device 109 to display the resultant image (step S18). In this case, the image of the space craft traveling in accordance of the duration of the out-of-bed state of the user is selected and presented.

Note that the management unit 115 can select an image of a mode to be different between time slots set by the first setting unit 113. For example, while the switching speed of the movie of the space craft is doubled for the time slot T1 as described above, the switching speed for the movie of the space craft may be tripled for the time slot T2 or the like. In this manner, the management unit 115 may select an image of the mode representing the special information of a pattern differing depending on a condition to be satisfied for the reward information set in advance.

Although in the case described in the embodiment, the estimation unit 110 estimates the user to be in the lying state or the out-of-bed state. However, the state of the user estimated by the estimation unit 110 may further include a walking state.

As described above, with the rehabilitation support system according to the second embodiment, when a user enters a specific state in a time or a time slot defined for the implementation of the rehabilitation, a mode of a spatiotemporally changing image that indicating the reward information is selected. Thus, the user can be motivated toward the rehabilitation in a time slot in which the user is particularly desired to work on the rehabilitation.

### Third Embodiment

Next, a third embodiment of the present invention will be described. In the following description, the same configurations as those in the first and the second embodiments described above will be denoted by the same reference signs and description thereof will be omitted.

In the cases described in the first and the second embodiments, the mode of the spatiotemporally changing image set in advance is stored in the storage unit 12 in association with the progress of the rehabilitation. In the third embodiment, the mode of the image to be presented as the rehabilitation support information is set for each user.

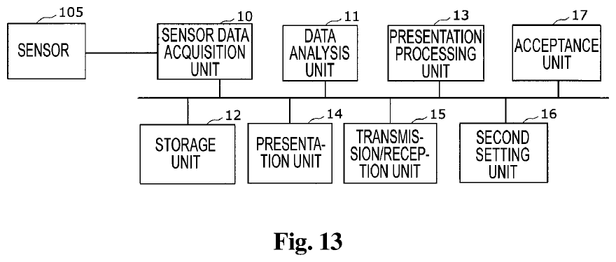

The configuration of the rehabilitation support system according to the third embodiment is different from those of the first and the second embodiments in that a second setting unit (setting unit) 16 and an acceptance unit 17 are further provided as illustrated in FIG. 13.

The second setting unit 16 sets the set time, which is a time slot defined for implementation of the rehabilitation, for each user. For example, the second setting unit 16 can set the set time for each user based on statistical data related to the rehabilitation and the record of the state of the user. The second setting unit 16 sets, for each user, the mode of the spatiotemporally changing item to be selected by the selection unit 111 in accordance with the state involved in the rehabilitation.

The acceptance unit 17 accepts an operation input for setting the mode of an item such as a spatiotemporally changing image, for each user. More specifically, the acceptance unit 17 accepts a mode of a spatiotemporally changing image for each user, selected in the set time for each user set by the second setting unit 16, and stores the mode in the storage unit (second storage unit) 12.

For example, the second setting unit 16 can associate in advance, the state of the user with a scene of a movie selected by the selection unit 111, in accordance with the progress of the rehabilitation of each user. Specifically, based on a record of activities during the past waking time of each user, an inactive time slot in which the rehabilitation is not performed by the user is estimated, the time slot is set as reward time (set time), and the mode of the spatiotemporally changing image that indicates the reward information is determined. Note that the past active period may be set in any manner to be set for each day, day of the week, and month, or the like depending on the characteristics of the rehabilitation or the user.

Now, a description will be given on an inactive time slot. For example, a case is considered where a user who is often in the lying state performs a physical rehabilitation such as getting out of bed or walking. In this case, the inactive time slot is the time slot, in the waking time of the user, in which the user is in the lying state, that is, is not performing the rehabilitation. Note that the inactive time slot is not limited to the time slot in which the user is in the lying state.

As another example, the time in which the user has been in the out-of-bed state or the walking state for example, at a certain time (noon for example) in a day, is assumed to be longer than the standard time, based on the history of the state of the user in that day. In this case, the user is expected to spend the inactive time, such as being in the lying state without performing the rehabilitation, for a long period of time in the afternoon of that day. Thus, for the afternoon of that day, the second setting unit 16 may set the reward time to be longer, so that the mode of the spatiotemporally changing image that represents the reward information is set to be selected more.

On the other hand, when the user is in the inactive for a period of time exceeding the standard time until the noon of the day, the active time, that is, the time during which the user performs the rehabilitation to be in the out-of-bed state or the walking state is expected to be longer in the after of the day than in the morning. Thus, the second setting unit 16 may not increase the reward time for the afternoon of that day, and set an image of a mode merely drawing attention to be selected when the inactive state in which the user is not performing the rehabilitation exceeds a certain period of time.

Note that the second setting unit 16 is not limited to a configuration of determining the reward time based on the time slot in which the user is inactive and setting the mode of the image selected such as the pattern of the movie. For example, the second setting unit 16 may similarly define the reward time based on the time slot in which the user is active, that is, in which the user is performing the rehabilitation. With this configuration, the user can be prompted to be more active by getting out of bed, walking, and the like, and can further be prompted to work out regularly whether during the rehabilitation or not.

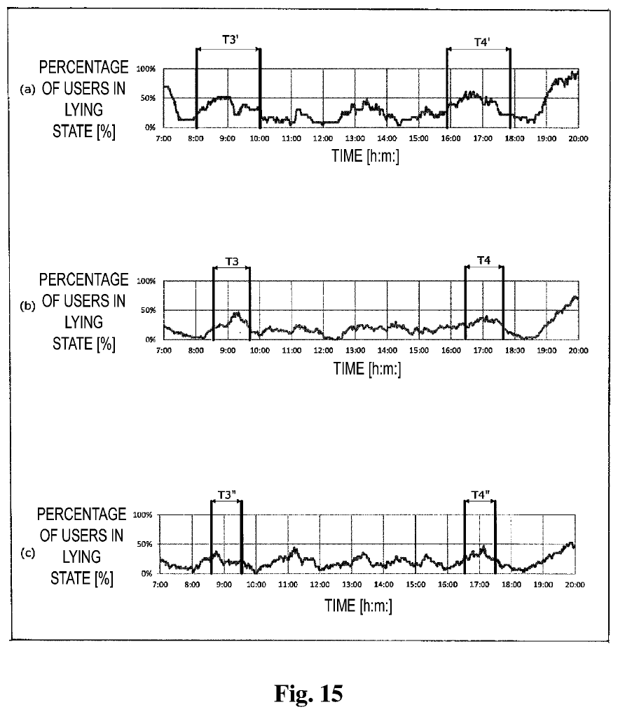

Furthermore, the second setting unit 16 may change the mode of the image selected, setting for the reward time, or the like, based on how much the function of each user has been rehabilitated in accordance with a rehabilitation plan for the user. For example, FIG. 15 illustrates the percentage of the users in the lying state for each value of a Functional Independence Measure (FIM). The FIM is an index for evaluating how much a patient (user) under rehabilitation can perform operations in their daily lives by him or herself. Although FIM is used herein as an example of an evaluation index indicating how much the user under the rehabilitation has rehabilitated his or her function, other evaluation indices such as a heart rate, body movement, elapsed time, stress value, and screening may also be used.

FIG. 15(b) illustrates the percentage of users in the lying state, with the FIM being equal to or more than 41 and equal to or less than 80, for each time. For the users with the FIM being equal to or more than 41 and equal to or less than 80, time slots T3 and T4 are set as the reward time. Thus, in the time slots T3 and T4, an image of the mode indicating the reward information prompting the user to perform the rehabilitation is presented as the rehabilitation support information.

For example, in FIG. 15(b), in the time slots T3 and T4, an image of a mode indicating the reward information for giving a reward to a user for being in the out-of-bed state or the walking state is selected and presented.

On the other hand, as in FIG. 15(a), a user with a value of the FIM being 50 or less is desired to more actively engage in the rehabilitation. Thus, for such users, time slots T3′ and T4′ longer than those for the users with the FIM being equal to or more than 41 and equal to or less than 80 are set as the reward time, meaning that the image of the mode indicating the reward information prompting the rehabilitation is selected and presented for a longer period of time.

As illustrated in FIG. 15(c), users with a value of the FIM being equal to or more than 81 is prompted to challenge a rehabilitation with a larger exercise load. For example, for such users, time slots T3″ and T4″ that are shorter for those for the users with the FIM being equal to or more than 41 and equal to or less than 80 are set as the reward time in which the image of the mode indicating the reward information is presented to a user who has performed the rehabilitation requiring the user to be in the walking state.

With the mode of the spatiotemporally changing item to be presented as the rehabilitation support information thus set in accordance with how much the user has rehabilitated his or her function, the functions of the user can be more effectively rehabilitated. Note that user information indicating how much the user has rehabilitated his or her function, such as the FIM value, for each user is stored in the storage unit 12 in advance.

The second setting unit 16 may refer to the value from the sensor 105 that measures information on the external environment of the user such as room temperature or humidity as described above, to set the mode of the spatiotemporally changing item that is selected by the selection unit 111. For example, a threshold may be set for the value of the room temperature or the like, and the image presented as the rehabilitation support information may be switched when the threshold is exceeded. Furthermore, an image of a mode that is more effective may be set to be selected in accordance with how much the user has rehabilitated his or her functions, with the reward time described above extended, shortened, provided, terminated, or the like when the threshold is exceeded. Note that in this case, the second setting unit 16 may use the sensor data from the plurality of sensors 105, and may use a value estimated based on the values of the sensor data.

Note that the second setting unit 16 may set only the reward time for each user. The reward time is a time slot in which the user is prompted to perform the rehabilitation as described above for example. Furthermore, the second setting unit 16 may set, for each user, only the mode of the spatiotemporally changing item that indicates the reward information selected when the user is performing the rehabilitation, during the common reward time set to the users.

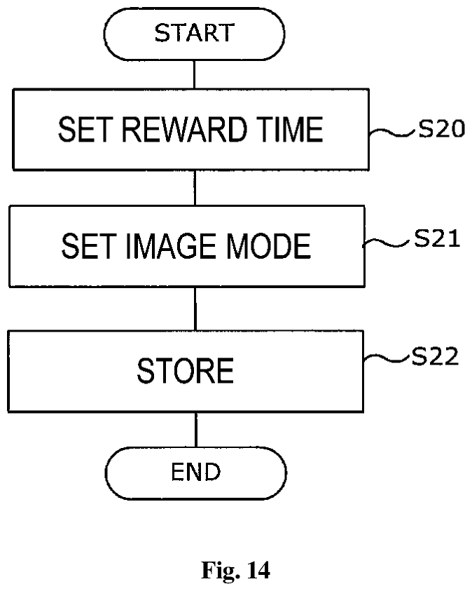

Next, an example of processing of setting the rehabilitation support information by the rehabilitation support system having the configuration as described above will be described with reference to a flowchart of FIG. 14. The following processing is executed independently from the processing in which the rehabilitation support system estimates the state of the user involved in the rehabilitation, and selects and presents the mode of the spatiotemporally changing image in accordance with the state of the user (FIG. 4 for example). For example, the processing may be executed before the processing described in FIG. 4. The processing may also be executed again after the processing described in FIG. 4 in accordance with how much the user has rehabilitated his or her function, to update the setting.

First, the second setting unit 16 sets a reward time, which is a time slot defined for the implementation of the rehabilitation, from the FIM value of the user stored in the storage unit 12 (step S20). For example, when the FIM value of the user is equal to or more than 41 and equal to or less than 80, the time slots T3 and T4 as illustrated in FIG. 15(b), are set as the reward time. As a result, when the user enters the out-of-bed state to perform the rehabilitation in these time slots T3 and T4, the image of the mode of giving a reward to the user is selected.

Next, the second setting unit 16 sets the mode of the spatiotemporally changing item selected by the selection unit 111 (step S21). More specifically, the image of the mode may be set to be selected to be different between the time slots T3 and T4 for example, based on the mode of the spatiotemporally changing item for each user accepted by the acceptance unit 17.

In this case, the second setting unit 16 may set a pattern in which the image is switched so that the space craft passes through the planet at a doubled speed, when the user is estimated to be in the out-of-bed state in the time slot T3 set as the reward time. Furthermore, the mode of the image of space craft traveling at a doubled speed may be set to be selected when the user is estimated to be in the walking state in the time slot T4. With this configuration, the user can be particularly prompted to take on the rehabilitation with a higher exercise load in the time slot T4.

A mode of an image indicating a process of a plant such as flower growing may be set to be selected in accordance with the progress of the rehabilitation of the user in the morning, and the image of the mode in which the space craft travels in space may be set to be selected in the afternoon. Furthermore, such setting of the type of the image may be changeable based on an input from the user.

Then, the second setting unit 16 stores the mode of the spatiotemporally changing image, such as the set pattern of the presentation of the image, in the storage unit 12 (step S22).

The estimation of the state of the user involved in the rehabilitation and the selection and the presentation of the mode of the image based on the estimation result as described in the first embodiment are performed (FIG. 4 and FIG. 12), by using the pattern of presentation of the rehabilitation support information set based on how much each user has rehabilitated his or her function through the procedure or the like described above.

As described above, with the rehabilitation support system according to the third embodiment, the presentation pattern of the rehabilitation support information is set in advance for each user, to increase the motivation toward the rehabilitation based on how much the function has been rehabilitated and characteristics of the user, which differ among users.

### Fourth Embodiment

Next, a fourth embodiment of the present invention will be described. In the following description, the same configurations as those in the first to third embodiments described above will be denoted by the same reference signs and description thereof will be omitted.

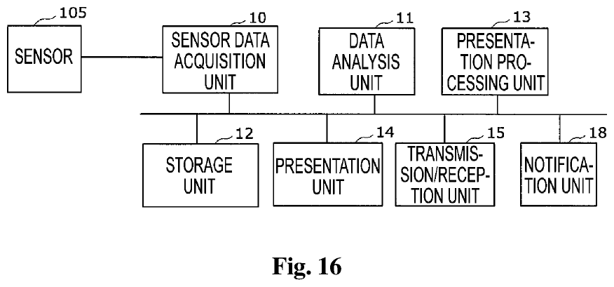

The rehabilitation support system according to the fourth embodiment has the configuration as in the second embodiment and further includes a notification unit 18 as illustrated in FIG. 16.

When the time or time slot defined for implementation of the rehabilitation set by the first setting unit 113 starts, the notification unit 18 notifies the user of the start of the time and the time slot. For example, the notification unit 18 can notify the user of the start by generating a notification in a format, such as text information, light, or a change in the color of the image, visually recognizable by the user. In this case, the notification unit 18 can cause the display device 109 to display the text information and the like.

Alternatively, the notification unit 18 notifies the user of the start by generating a notification in a format that can be audibly recognized by the user, such as sound, that is, a change in or muting of the sound for example. In this case, the notification unit 18 outputs the notification from a speaker or the like. Furthermore, the notification unit 18 may perform the notification by generating a notification in a format that is tactilely recognizable by the user such as vibration, electric shock, and heat. In this case, the notification unit 18 outputs a notification from an operating device such as an oscillator (not illustrated).

The notification unit 18 may send a notification to a terminal (not illustrated) connected to the communication interface 104 through the communication network NW. In such a case, the notification may be issued to a person other than the user, such as a caregiver, and the person may notify the user of the start time for example.

As described above, with the rehabilitation support system according to the fourth embodiment, the user is notified of the start of a time or time slot defined for the implementation of the rehabilitation, such as the reward time, so that the user performing the rehabilitation can be more reliably supported.

### Fifth Embodiment

Next, a fifth embodiment of the present invention will be described. In the following description, the same configurations as those in the first to fourth embodiments described above will be denoted by the same reference signs and description thereof will be omitted.

In the case described in the first embodiment, the estimation unit 110 estimates the state of the user such as the out-of-bed state for example, and based on the estimation result, the selection unit 111 selects the mode of the spatiotemporally changing image. In the fifth embodiment, a selection unit 111B selects a feedback set in advance, in accordance with a plurality of different states of the user involved in the rehabilitation.

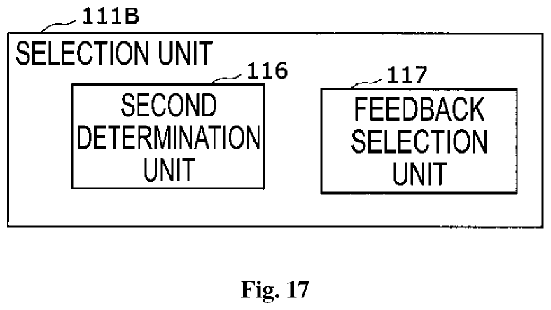

FIG. 17 is a block diagram illustrating a configuration of the selection unit 111B included in the rehabilitation support system according to the present embodiment. The other configuration of the rehabilitation support system according to the present embodiment is the same as that in the first embodiment.

The estimation unit 110 estimates a plurality of different states of the user involved in the rehabilitation. For example, when the user who is often in the lying state performs rehabilitation involving getting out of bed and walking, the estimation unit 110 can estimate the lying state, the out-of-bed state, and the walking state for the user. Any of these states of the user is estimated based on the acceleration data on the user measured by the sensor 105 including the acceleration sensor acquired by the sensor data acquisition unit 10 for example. The estimation unit 110 may estimate the state of the user at a fixed interval.

The selection unit 111B selects the spatiotemporally changing mode of the item such as an image, in accordance with the state of the user estimated by the estimation unit 110. The selection unit 111B includes a second determination unit 116 and a feedback selection unit 117.

The second determination unit 116 determines that the state of the user estimated by the estimation unit 110 has transitioned to a state of the user set in advance. For example, the second determination unit 116 determines that the state of the user has transitioned to a state of the user set in advance, for the sake of effectiveness of the rehabilitation. More specifically, the lying state, the out-of-bed state, and the walking state estimated by the estimation unit 110 is determined to have transitioned to a state set in advance. For example, transition from the lying state to the out-of-bed state or from the out-of-bed state to the lying state or the like is determined.

For example, it is assumed that an action involving a higher rehabilitation effect is desirable for the user who is often in the lying state when he or she performs rehabilitation such as getting out of bed or walking. The second determination unit 116 determines that, for example, the user who has been taking an action involving a lower rehabilitation effect has started taking an action involving a higher rehabilitation effect, or the user who has been taking an action involving a higher rehabilitation effect has started taking an action involving a lower rehabilitation effect.

Specifically, the three different states estimated by the estimation unit 110 that are the lying state, the out-of-bed state, and the walking state are expected to involve a larger exercise load and thus higher rehabilitation effect in this order. Thus, when the estimation unit 110 estimates the state of the user at a fixed interval for example, the second determination unit 116 compares the immediately preceding state of the user with the current state of the user based on the exercise load, and outputs the result of the comparison. In this case, the second determination unit 116 can determine that the state of the user has transitioned to a state with a lower rehabilitation effect and that the state of the user has transitioned to a higher rehabilitation effect.

The feedback selection unit 117 selects a feedback set in advance, to the user based on the result of the determination by the second determination unit 116. The feedback selection unit 117 selects a positive feedback among the feedbacks stored in advance in the storage unit 12, for example, when the state of the user transitions to a state with a higher rehabilitation effect. On the other hand, the feedback selection unit 117 selects a negative feedback in the storage unit 12, when the state of the user transitions from the state with a high rehabilitation effect to a state with a low rehabilitation effect.

What kind of feedback is selected by the feedback selection unit 117 based on the result of the determination by the second determination unit 116 can be set in accordance with the action the user performing the rehabilitation is prompted to take. For example, as described above, negative feedback can be selected for a transition from to a state with a higher exercise load in case where the user is prompted to be in a resting state, in addition to the case where the user is prompted to an activity with a larger exercise load for the sake of higher rehabilitation effect.

Now, the feedback selected by the feedback selection unit 117 will be described in more detail. Feedback is information indicating an evaluation for a change in the action taken by the user, and is provided to the user to be a chance for improving the situation of the user under the rehabilitation. Furthermore, the feedback is the mode of the spatiotemporally changing item. The feedback selection unit 117 can adjust the length of time for switching a spatiotemporally changing image, or may select a special image, sound, text, vibration, heat, light, wind, stimulation, and the like.

The positive feedback is information indicating to the user performing the rehabilitation that the user's current efforts are going the right way, to praise the action of the user, namely, the effort of the user performing the rehabilitation so that he or she can be more motivated. The feedback selection unit 117 can use the positive feedback to shorten the time for switching the spatiotemporally changing image, for example.

The feedback selection unit 117 can for example, select text information or voice saying “Excellent job XX! Keep up the good work” as the positive feedback.

On the other hand, the negative feedback is information that notifies the user performing the rehabilitation that how he or she is handling the rehabilitation is not favorable in terms of rehabilitation effect, to warn the user. Such negative feedback is information for warning the user who is not performing the rehabilitation or demotivated toward the rehabilitation, and prompt him or her to work on the rehabilitation for example.

For example, the feedback selection unit 117 can use the negative feedback to expand the time for switching the spatiotemporally changing image.

The feedback selection unit 117 can select as the negative feedback, text information or voice saying “Come on XX, I know you can do more” or the like for example. Still, the negative feedback selected should not be the image of a mode that is too negative that it could even discourage the user to work on the rehabilitation.

The feedback selected by the feedback selection unit 117 based on the result of the determination by the second determination unit 116 may also be selected when other conditions are satisfied. For example, a condition may be set in terms of time. In this case, the feedback selection unit 117 may select a positive or negative feedback when the state of the user continues for a certain period of time, such as a minute or more, after the transition of the state of the user. Thus, the user can accept the feedback without getting annoyed, even when the state of the user frequently transitions.

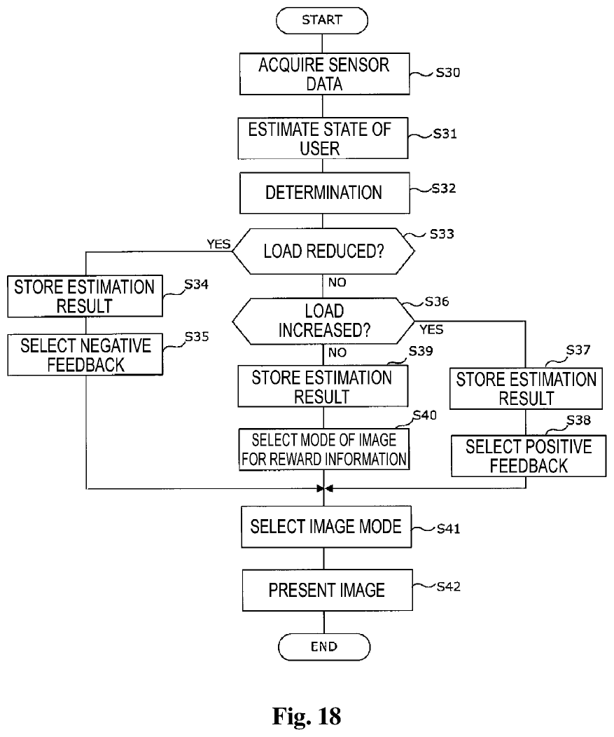

Next, the operation of the rehabilitation support system according to the present embodiment configured as described above will be described with reference to a flowchart of FIG. 18. First, the following processing is executed in a state where the sensor 105 is attached to the user. Furthermore, a specific example where the user who is often in the lying state is prompted to perform the rehabilitation of getting out of bed and walking will be described.

The sensor data acquisition unit 10 acquires the biometric information on the user measured by the sensor 105 via the transmission/reception unit 15 (step S30). The acquired biometric information is accumulated in the storage unit 12. Note that the sensor data acquisition unit 10 can execute processing of remove the noise in the biometric information acquired, and converting the analog signal of the biometric information into a digital signal.

Next, the estimation unit 110 estimates the state of the user based on the biometric information on the user acquired by the sensor data acquisition unit 10 (step S31). Specifically, the estimation unit 110 estimates that the user is in the lying state, the out-of-bed state, or the walking state from the data indicating the acceleration of the user acquired by the sensor data acquisition unit 10. The estimation unit 110 estimates the state of the user at the set time interval. The state of the user estimated is stored in the storage unit 12 together with the time information.

Next, the second determination unit 116 detects that the transition of the state of the user, and determines whether the user has transitioned from the immediately preceding state to a state requiring a larger exercise load, or to a state requiring a smaller exercise load (step S32).

More specifically, when state of the user transition from the out-of-bed state as the immediately preceding state to the lying state, a result of determination indicating the transition to a state requiring a smaller exercise load is output (step S33: YES), and the current state of the user is stored in the storage unit 12 (step S34).

Next, the feedback selection unit 117 selects the negative feedback among the feedbacks stored in the storage unit 12 (step S35). For example, the feedback selection unit 117 can select the mode of the spatiotemporally changing image that is displayed for a long period of time until it is switched.

Then, the selection unit 111A selects the mode of the spatiotemporally changing image, based on the history of the out-of-bed state of the user (step S41). Specifically, the selection unit 111A selects the mode of the image of the space craft launched from the earth and traveling through the planets, based on the history of the out-of-bed state of the user for example.

Then, the presentation unit 14 causes the display device 109 to display the image of the mode selected by the selection unit 111A and the negative feedback selected by the feedback selection unit 117 (step S42). For example, the presentation unit 14 may display, together with the image of the space craft reaching Mars, an image of a mode requiring a longer time to reach the next planet, Jupiter.

On the other hand, when the immediately preceding state of the user estimated in step S31 has transitioned to a state requiring a larger exercise load (step S33; NO, step S36: YES), the second determination unit 116 stores the estimation result in the storage unit 12 together with the time information (step S37). Then, the feedback selection unit 117 selects the positive feedback among the feedbacks stored in the storage unit 12 (step S38).

For example, a mode of a spatiotemporally changing image that is switch in a shorter period of time may be selected by the feedback selection unit 117 as the positive feedback, when the lying state of the user that is the immediately preceding state transitions to the out-of-bed state or the walking state.

Then, the selection unit 111B selects the mode of the spatiotemporally changing image, based on the history of the out-of-bed state of the user (step S41). Then, the presentation unit 14 causes the display device 109 to display an image of the mode selected in step S41 and positive feedback selected in step S38 (step S42). For example, the presentation unit 14 may display, for the image of the mode in which the space craft reaches Jupiter from Mars, an image of a mode requiring a shorter period of time for the space craft to reach Jupiter.

When there is no transition in the state of the user (step S33: NO, step S36: NO), the estimated result is stored in the storage unit 12 (step S40), and the selection unit 111A selects the mode of the spatiotemporally changing image that indicates the reward information (step S40). The selection unit 111A selects an image of the mode of giving a reward to the user, to prompt the user to change his or her activities to those requiring a larger exercise load. For example, an image of a mode may be selected that indicates that a point is given to the user if he or she performs a rehabilitation requiring a larger exercise load.

Then, the selection unit 111B selects the mode of the spatiotemporally changing image, based on the history of the out-of-bed state of the user (step S41). Then, the presentation unit 14 causes the display device 109 to display the image of the mode indicating the reward information together with the mode of the spatiotemporally changing image selected.

As described above, with the rehabilitation support system according to the present embodiment, positive or negative feedback is selected and presented in response to a transition of the state of the user. Thus, the activity can be set for the user more in detail, to prompt the user to challenge an activity requiring a larger exercise load.

Note that the described embodiments can be implemented in combination. For example, the second embodiment and the fifth embodiment may be combined. An example is considered where a certain state of the user is estimated in the set time which is the time slot defined for implementation of the rehabilitation. In this case, a configuration may be adopted in which spatiotemporally changing images of different modes are selected for respective exercise loads, as the reward information. For example, when the user transitions to the walking state for the first time in the set time slot, the mode in which the switching speed of the image is doubled is selected as the mode of the spatiotemporally changing image that indicates the reward information. Furthermore, a mode may be selected in which the image switching speed increases to be tripled, quadrupled, and so on as the duration of the walking state that is a state requiring a larger exercise load, increases.

The rehabilitation support systems according to the second to the fifth embodiments described above may be achieved by the sensor terminals 200a and 200b, the relay terminal 300, and the external terminal 400 illustrated in FIG. 5, FIG. 8, and FIG. 9 as in the first embodiment.

Furthermore, in the above description, the data analysis unit 11 is included in the relay terminal 300 in the rehabilitation support system achieved by the sensor terminals 200a and 200b, the relay terminal 300, and the external terminal 400. Alternatively, the data analysis unit 11 may be included in the sensor terminals 200a and 200b or the external terminal 400.

The functions (the estimation unit 110 and the selection unit 111) of the data analysis unit 11 may be distributed among the sensor terminals 200a and 200b, the relay terminal 300, and the external terminal 400 to be implemented.

Although the embodiment of the rehabilitation support system, the rehabilitation support method, and the rehabilitation support program of embodiments of the present invention have been described above, the present invention is not limited to the described embodiments, and various types of modification that can be conceived by a person skilled in the art can be made within the scope of the invention .

### REFERENCE SIGNS LIST

10, 202 Sensor data acquisition unit

11, 303 Data analysis unit

12 Storage unit

13, 403 Presentation processing unit

14, 404 Presentation unit

15 Transmission/reception unit

110 Estimation unit

111 Selection unit

101 Bus

102 Processor

103 Main storage device

104 Communication interface

105, 201 Sensor

106 Auxiliary storage device

107 Timepiece

108 Input/output device

109 Display device

200a, 200b Sensor terminal

300 Relay terminal

400 External terminal

203, 302, 402 Data storage unit

204, 304 Transmission unit

301, 401 Reception unit

1.-8. (canceled)

9. A rehabilitation support system comprising:
a sensor data acquirer configured to acquire biometric information on a user measured by a sensor;
an estimator configured to estimate a state of the user based on the biometric information;
a first determiner configured to determine whether the state of the user estimated by the estimator has occurred within a set time, the set time comprising a time slot defined for implementation of a rehabilitation;
a first storage device configured to store a mode of a spatiotemporally changing item;
a selector configured to select the mode of the spatiotemporally changing item, in accordance with a result of the determination by the first determiner; and
a presenter configured to present the mode of the spatiotemporally changing item selected by the selector.

10. The rehabilitation support system of claim 9, wherein the presenter comprises a display device configured to display an image representing the mode of the spatiotemporally changing item selected by the selector.

11. The rehabilitation support system of claim 9 further comprising:
a setter configured to set the set time for each user of a plurality of users, based on statistical data on implementation of the rehabilitation; and
a second storage device configured to store the set time defined for each user,
wherein the first determiner is configured to determine whether the state of the user estimated by the estimator has occurred within the set time defined for the user.

12. The rehabilitation support system of claim 11 further comprising:
an accepter configured to accept an operation input for setting the mode of the spatiotemporally changing item for each user,
wherein the first storage device is configured to store the mode of the spatiotemporally changing item set for each user.

13. The rehabilitation support system of claim 9 further comprising:
a notifier configured to issue a notification indicating start of the set time.

14. The rehabilitation support system of claim 9, wherein:
the estimator is configured to periodically estimate the state of the user; and
the rehabilitation support system further comprises:
a second determiner configured to determine that the state of the user has transitioned to a state of the user set in advance; and
a feedback selector configured to select a feedback to the user, in accordance with a result of the determination by the second determiner,
wherein the state of the user comprises a plurality of different states corresponding to different exercise loads, and
wherein the feedback selector selects as the feedback, the mode of the spatiotemporally changing item.

15. A rehabilitation support method comprising:
acquiring biometric information on a user measured by a sensor;
estimating a state of the user based on the biometric information;
determining whether the state of the user has occurred within a set time, the set time comprising a time slot defined for implementation of a rehabilitation;
selecting a mode of a spatiotemporally changing item stored in a first storage device, in accordance with a result of the determining whether the state of the user has occurred within the set time; and
presenting the mode of the spatiotemporally changing item.

16. The rehabilitation support method of claim 15, wherein presenting the mode of the spatiotemporally changing item comprises displaying an image representing the mode of the spatiotemporally changing item.

17. The rehabilitation support method of claim 15 further comprising:
setting the set time for each user of a plurality of users, based on statistical data on implementation of the rehabilitation;
storing the set time defined for each user; and
determining whether the state of the user estimated by the estimator has occurred within the set time defined for the user.

18. The rehabilitation support method of claim 17 further comprising:
accepting an operation input for setting the mode of the spatiotemporally changing item for each user; and
storing the mode of the spatiotemporally changing item set for each user.

19. The rehabilitation support method of claim 15 further comprising:
issuing a notification indicating start of the set time.

20. The rehabilitation support method of claim 15 further comprising:
periodically estimating the state of the user;
determining that the state of the user has transitioned to a state of the user set in advance; and
selecting a feedback to the user, in accordance with a result of the determining that the state of the user has transitioned,
wherein the state of the user comprises a plurality of different states corresponding to different exercise loads, and
wherein selecting the feedback comprises selecting, as the feedback, the mode of the spatiotemporally changing item.

21. A non-transitory computer readable storage medium storing a rehabilitation support program for execution by a processor, the rehabilitation support program comprising instructions for:
acquiring biometric information on a user measured by a sensor;
estimating a state of the user based on the biometric information;
determining whether the state of the user has occurred within a set time, the set time comprising a time slot defined for implementation of a rehabilitation;
selecting a mode of a spatiotemporally changing item stored in a first storage device, in accordance with a result of the determining whether the state of the user has occurred within the set time; and
presenting the mode of the spatiotemporally changing item.

22. The non-transitory computer readable storage medium of claim 21, wherein the instructions for presenting the mode of the spatiotemporally changing item comprises instructions for displaying an image representing the mode of the spatiotemporally changing item.

23. The non-transitory computer readable storage medium of claim 21, wherein the rehabilitation support program further comprises instructions for:
setting the set time for each user of a plurality of users, based on statistical data on implementation of the rehabilitation;
storing the set time defined for each user; and
determining whether the state of the user estimated by the estimator has occurred within the set time defined for the user.

24. The non-transitory computer readable storage medium of claim 23, wherein the rehabilitation support program further comprises instructions for:
accepting an operation input for setting the mode of the spatiotemporally changing item for each user; and
storing the mode of the spatiotemporally changing item set for each user.

25. The non-transitory computer readable storage medium of claim 21, wherein the rehabilitation support program further comprises instructions for:
issuing a notification indicating start of the set time.

26. The non-transitory computer readable storage medium of claim 21, wherein the rehabilitation support program further comprises instructions for:
periodically estimating the state of the user;
determining that the state of the user has transitioned to a state of the user set in advance; and
selecting a feedback to the user, in accordance with a result of the determining that the state of the user has transitioned,
wherein the state of the user comprises a plurality of different states corresponding to different exercise loads, and
wherein the instructions for selecting the feedback comprises instructions for selecting, as the feedback, the mode of the spatiotemporally changing item.
